(19) United States
(12) Patent Application Publication
(10) Pub. No.: US 2021/0015425 A1
(43) Pub. Date: Jan. 21, 2021

(54) PORTABLE HYDRATION MONITORING DEVICE AND METHODS

(71) Applicant: GIDEON HEALTH, INC., Los Angeles, CA (US)

(72) Inventors: David E. CLIFT-REAVES, Austin, TX (US); Dustin M. FRECKLETON, Austin, TX (US); Paulo E. XAVIER DA SILVEIRA, Boulder, CO (US); Byron P. OLSON, Boone, IA (US); Nithin O. RAJAN, Austin, TX (US)

(73) Assignee: GIDEON HEALTH, INC., Los Angeles, CA (US)

(21) Appl. No.: 16/968,843

(22) PCT Filed: Feb. 9, 2019
(86) PCT No.: PCT/US19/17399

§ 371 Date: Aug. 10, 2020

Related U.S. Application Data
(60) Provisional application No. 62/628,750, filed on Feb. 9, 2018.

(51) Int. Cl. A61B 5/00; A61B 5/026; A61B 5/024; A61B 5/053; A61B 5/08; G16H 50/20; G16H 50/30

(52) U.S. Cl. CPC A61B 5/4875; A61B 2562/0233; A61B 5/02416; A61B 5/0537; A61B 5/6826; A61B 5/02438; A61B 5/02405; A61B 5/0816; A61B 5/746; A61B 5/681; G16H 50/20; G16H 50/30; A61B 2503/10; A61B 2562/0219; A61B 2562/0247; A61B 5/0261

(57) ABSTRACT

A method operable to monitor hydration including pressing a finger of a user against a finger cradle of a device, creating a finger press; sensing, via at least one sensor, a finger presence; providing an alarm indicating a pressure to apply; emitting, via at least one electromagnetic emitter, radiation through the finger; detecting, via at least one electromagnetic detector, radiation through the finger; verifying, via a synchronization line, that the electromagnetic radiation is applied synchronously with one or more finger presses; converting, via at least one analog-to-digital converter, the radiation detected by the electromagnetic detector into digital information; and converting, via a processing unit, the finger pressure data and the detected electromagnetic radiation data into a hydration level using an algorithm stored on the processing unit; and conveying the hydration level to at least one alarm unit coupled with the device.

### CROSS-REFERENCE TO RELATED APPLICATION

This application claims priority to U.S. Provisional Patent Application 62/628,750, which was filed in the U.S. Patent and Trademark Office on Feb. 9, 2018, all of which is incorporated herein by reference in its entirety for all purposes.

### FILED

The present disclosure relates to a portable device for hydration monitoring and methods for use thereof.

### BACKGROUND

Numerous monitoring devices are currently available in the market configured to track various aspects of a user's health. Such devices can be capable of tracking factors such as a user's heart rate, activity throughout a defined period, or steps taken throughout a defined period. Such devices can be wearable and in some examples can be integrated into watches.

### BRIEF DESCRIPTION OF THE DRAWINGS

Implementations of the present technology will now be described, by way of example only, with reference to the attached figures, wherein:

FIG. 1 illustrates a schematic diagram of a device for monitoring hydration in a user in accordance with the present disclosure;

FIG. 2 illustrates an example of a synchronization method for a hydration monitoring device in accordance with the present disclosure;

FIG. 3A is a flowchart illustrating a method for determining wellness goals in accordance with the present disclosure;

FIG. 3B is a flowchart illustrating a method for assisting a user in attaining a wellness goal in accordance with the present disclosure;

FIG. 4 is a graph illustrating the power spectral density of a well hydrated user in accordance with the present disclosure;

FIG. 5 is a graph illustrating the power spectral density of a user having a 1.97% dehydration level in accordance with the present disclosure;

FIG. 6 is a graph illustrating the skin turgor signal as measured in a user who is well hydrated in accordance with the present disclosure;

FIG. 7 is a graph illustrating the skin turgor signal as measured in a user having a 1.97% dehydration level in accordance with the present disclosure; and

FIG. 8 is a graph illustrating the variation in the electromagnetic signal obtained from a user as finger pressure is varied on the hydration monitoring device in accordance with the present disclosure.

### DETAILED DESCRIPTION

The present disclosure relates to a portable, non-invasive monitoring device and methods for spot-checking measurements of a hydration state of a user. The monitoring device and methods for use thereof can find application not only in mobile devices, smartphones, and wearable devices, but also in medical applications wherein it can be critical to determine the hydration state of an individual, without a previous hydration history.

The term “finger press” as used herein can consist of step responses, which are inherently broadband. As the finger of a user is pressed and depressed against a finger cradle, blood flows into and out of the capillaries in the extremities of the user's finger. This flow, however, can be non-uniform and the temporal response can vary as a function of the hydration state of the user.

The term “hydration state” as used herein refers to the present level of hydration in a user. A euhydradrated state refers to a normal state of body water content.

As used herein, the term “sufficiently broadband” refers to a finger press that is sufficient variable in time for the device to collect the necessary data for use in the described methods.

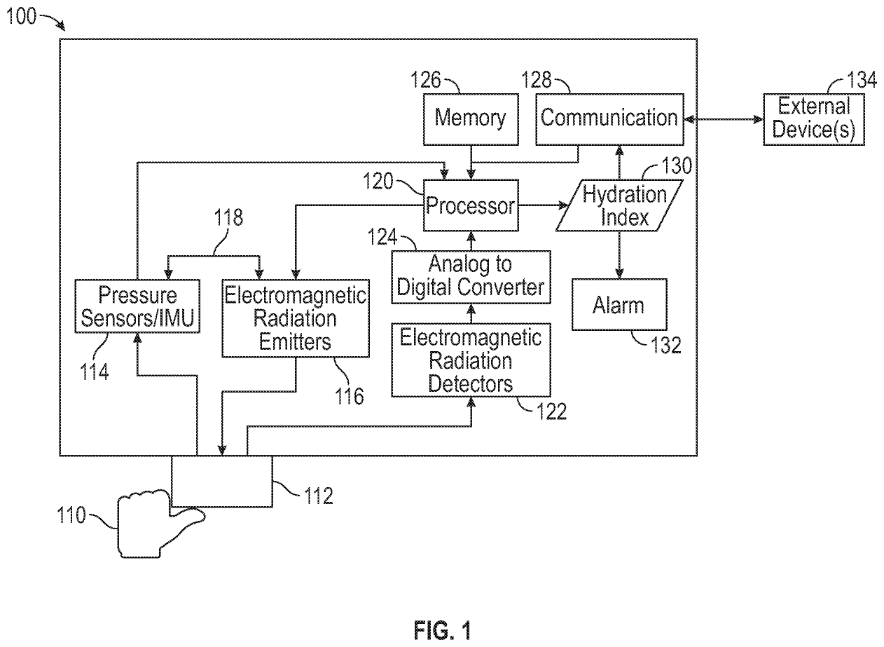

FIG. 1 is a schematic diagram illustrating an apparatus described herein which can be used for portable, non-invasive monitoring of hydration of a user. Specifically, FIG. 1 shows an exempe of a portable hydration monitoring device 100 capable of assessing the hydration state of a user. For example, the hydration state can be evaluated by having a user press his/her finger 110 against a finger cradle 112 coupled with the device 100. As used herein, the term “finger” refers to any of the five phalanges, including the thumb. Additionally, the finger can be on either right or left hand. As used herein, the term “finger cradle” can include any suitable contact point, the finger cradle can be coupled with a pressure sensor to which a finger can be pressed. In at least one example, the finger cradle be located on the top of the device. In an alternative example, the finger cradle can be a protuberance from the device. In at least one example, the finger cradle can include an indentation for which a user can place their finger. In at least one embodiment, the finger cradle can also include a flat surface embedded into the device; the flat surface can include, but is not limited to, a glass plate, a plastic plate, and combinations thereof. In at least one example, the flat surface of the finger cradle can be similar to those frequently used in fingerprint readers. The finger cradle can be made of one or more materials including, but not limited to, plastic, glass, metal, and combinations thereof.

The device 100 can further include one or more pressure sensors, such as inertial measurement units (IMUs) 114, one or more electromagnetic radiation emitters 116, and one or more electromagnetic radiation detectors 122; each of each of the above elements can be coupled with the finger cradle 112. The pressure sensors/IMUs 114 and the electromagnetic radiation emitters 116 can be coupled providing a synchronization line 118. Additionally, each of the pressure sensors/IMUs 114, electromagnetic radiation emitters 116, and electromagnetic radiation detectors 122 can be coupled with a processor 120. The one or more electromagnetic radiation detectors 122 can be coupled with the processor 120 via an analog-to-digital converter 124, the converter 124 allowing data obtained by the electromagnetic radiation detectors to be readable by the processor. The processor 120 can be further coupled with a memory 126 capable of storing historical user data, a hydration index 130 configured to store hydration data, and a communication unit 128 configured to send and receive information between the device 100 and an external device 134. The hydration index 130 can also be coupled with the communication unit 128 and can be configured to issue an alarm 132 when one or more predetermined circumstances are met. Such circumstances can include, but are not limited to, a decrease in the hydration state of the user. The alarm can include, but is not limited to, a visual alarm, an audible alarm, a display indicating a user's hydration state, an audible message indicating a user's hydration state, and the like. The alarm 132 can then be transmitted from the device 100 to the external device 134 via communication unit 128 in order to provide a user with data relating to the one or more circumstances.

In at least one example, the device can obtain data by measuring both turgor and capillary refill time of the skin in the finger of a user and evaluate said measurements as a function of applied pressure . For example, such measurements can be taken by interrogating the tissue of a finger with electromagnetic radiation, which can be emitted from the device towards a finger via the finger cradle as described above. The timing and amount of radiation reflected back can be detected via the electromagnetic detectors in the device, as described with respect to FIG. 1. In order to obtain accurate measurements, several elements of the interrogation must be tracked including, but not limited to, the amount of radiation reflected, the spectrum of the radiation reflected, and the timing with respect to the finger pressure against the finger cradle, such as the time between finger presses. Such information is critical to performing the analysis described herein.

In at least one example, the electromagnetic radiation can include one or more near infrared lights. For example, the electromagnetic radiation emitters can be implemented via one or more light emitting devices (LEDs); the electromagnetic radiation detectors can be implemented via one or more silicon photodiodes. In at least one example, multiple LEDs can be used; the LEDs can be used to emit light in a wavelength range from 600 nm to 1000 nm. The use of a wavelength between 600 nm and 1000 nm can allow for a relatively high transmission of light through biological tissue, such as the tissue of a finger. Additionally, the use of silicon photodiodes as the electromagnetic radiation detectors can allow for relatively high photoresposivity and can lower the overall cost of the light source. For example, the LEDs can emit light at a centroid and/or mean wavelengths of from about 650 nm to about 950 nm, to allow for the detection of hemoglobin chromophores, such as oxyhemoglobin and deoxyhemoglobin, and the detection of their sum, total hemoglobin (tHb).

It should be noted that the present disclosure is not limited to optical monitoring. In an alternative example, the tissue could be monitored using methods including, but not limited to, galvanic skin response, electrodermal activity, skin conductance response, sympathetic skin response, skin conductance level, the bioimpedance of tissue, and combinations thereof. For example, the electromagnetic radiation emission could include the emission of electric signals ranging in frequency from the long waves (approximately 100 Hz) to short wave radio frequencies (approximately 250 MHz).

As described above, the device can include a processor which can be used to control each of the sub-components described above. Additionally, the processor can be used to execute code capable of converting the signals received at the electromagnetic radiation detectors into a hydration index, the hydration index can be indicative of the hydration state of the user. In addition, the device can be equipped with a memory which can store instructions and execute a code via the one or more processors, the memory can further store data collected from the electromagnetic radiation detectors, and can be coupled with an alarm unit. The alarm unit can be configured to issue an alert informing the user that their hydration level has reached a level that is not considered ideal, for example, if their hydration level is too low. In at least one example, the alarm unit can further be used to provide the user with information regarding their hydration index. Furthermore, in at least one example, the alarm unit can perform additional tasks including, but not limited to, communicating a request for the user to perform a specific measurement, providing the user with feedback regarding the correct placement of the user's finger on the finger cradle, and providing the user with feedback regarding the correct use of the device. The alarm unit can further include a display, an audible alarm, or both a display and an audible alarm. For example, the alarm issued by the alarm unit can be, but is not limited to, a visual alarm, an audible alarm, a display or notification indicating hydration information for the user, an audible message indicating a hydration state for a user, as well as combinations of two or more of the above described alarms.

As described above, the device can also be equipped with a communication unit which can be operable to transmit information and data to from the device to one or more external devices. For example, the communication unit can transmit data using wired or wireless communication (such as, Bluetooth, Wi-Fi, ZigBee). The data can be transmitted to a user via an alarm sent to their mobile device, including, but not limited to, a smartphone, a personal computer (PC), a tablet, a laptop, or combinations thereof. Furthermore, if the user's mobile device has a calendar application, the communication unit can generate reminders in the calendar on the smartphone, laptop, PC or tablet. Such reminders can be used to inform the user not only when to consume fluids, but also how much fluids the user needed to drink in order to reach a desired hydration level.

In at least one example, the device as described above can be integrated directly into a wearable device, a watch, a smartphone, a cellphone, a tablet, a computer, a laptop, or combinations thereof. Where the device is integrated in a wearable device, the device can be worn such that it is coupled with the user in a location including, but not limited to, the user's wrist, arm, chest, head, neck, waist, leg, and/or abdomen.

In at least one example, the pressure sensor can be a pressure plate. In an alternative example, the pressure sensor can be a capacitive force sensor. In yet another alternative example, the device pressure sensor can be, a piezoelectric transducer coupled with a transimpedance amplifier and an analog-to-digital converter. The pressure sensor can be enabled by an inertial measurement unit (IMU) coupled with the pressure sensor and containing an accelerometer, a gyroscope, a motion sensor, and combinations thereof. In at least one example, the IMU can be, for example, an Invensense MPU-6000. While the recited IMU may not be able to precisely measure a pressure applied to a pressure sensor, it can be used to accurately and quickly detect finger motion, and the IMU output signal can be easily synchronized with the electromagnetic radiation detectors.

In at least one example, the electromagnetic radiation emitters and the electromagnetic radiation detectors can be integrated into a single package. For example, the electromagnetic radiation emitter and detector can be packaged together in a module including one or more LEDs, one or more light-blocking elements, and one or more anti-reflection coated photodiodes. Such combined electromagnetic radiation emitter/detectors can optimize the detection of specific wavelengths and increase the reliability of the device, while also providing a reduced part count and a simplified fabrication of the device. Furthermore, the analog-to-digital converter can also be integrated with additional elements, such as a part of an analog front end (AFE) device which can also incorporate the current drivers used for the one or more LEDs. In an alternative example, the AFE could be included in the device as an individual component. In yet an alternative embodiment, the optical package and the AFE can be integrated into a single component.

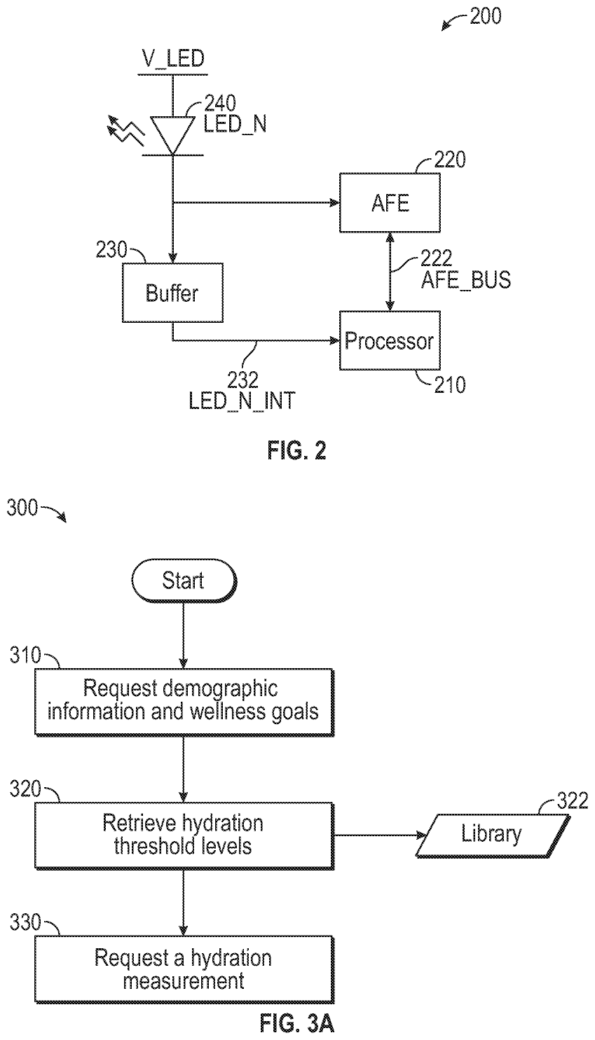

Integration of certain parts of the device, as described herein can further simplify the synchronization between the parts, as illustrated in FIG. 2. Specifically, FIG. 2 illustrates the synchronization between an LED and the processor. As illustrated, a device 200 can include a processor 210 coupled with an AFE 220 via an AFE_BUS 222, and a buffer 230 via an LED_N_INT 232. The device 200 can further include one or more LEDs which can be coupled with both the buffer 230 and the AFE 220. In the example, the synchronization between the elements can be controlled via the processor, which can communicate with the AFE, which can the electromagnetic radiation emitters and electromagnetic radiation detectors, through the AFE_BUS. In at least one example, the AFE_BUS can be an I2C or serial peripheral interface (SPI). The AFE_BUS data can be used to determine a first-in-first-out (FIFO) stack. In order for the time to be accurately recorded, a start time must be triggered via an external input. In at least one alternative example, the time of the light emission must be accurately recorded by the processor and correlated to a sufficiently oversampled force signal. Then, after the samples in the FIFO are recorded, they can be synchronized with respect to the force samples based on the recorded firing times.

The external input can be implemented by a series of signals, N, where N denotes the number of LEDs which are used within the device. For example, in at least one configuration the value of N can be 2. As such, LED_N_INT represents the Nth interrupt signal for the processor which is generated via the LED's emissions. In at least one example, it each of the LED_N_INT signals can be combined into a single signal through an exclusive Boolean gate (such as an OR gate), before being entered into the processor. Additionally, circuitry can be included which slows the rise of the signal, this can allow the processor to catch whether the emission is below the trigger level. In that case, the buffer can actively drive the signal down, after which it can take a longer period to rise back up, allowing the processor time to trigger the interrupt. In an alternative, a comparator can be used as well as a latch to latch the signal low until enough triggering signal has accumulated, thus providing the processor with an alternate synchronization signal.

Furthermore, in at least one embodiment, the above described synchronization can be achieved by using the one or more pressure sensors/IMUs 114 to indicate to the processor when a finger is pressed to, or the pressure is released from, the pressure sensor. In said example, the synchronization process can allow the processor to measure the time difference between multiple finger presses and changes in the digitized electromagnetic signal detected. In at least one embodiment, the time difference can be recorded using an internal clock coupled with the processor of the device.

In an alternative embodiment, the synchronization process can take place directly between the one or more pressure sensors/IMUs 114 and the one or more electromagnetic radiation emitters 116. In said example, the synchronization process can use the detection of a finger press and/or release at the one or more pressure sensors/IMUs 114 in order to control the emission timing from the electromagnetic radiation emitter, such that electromagnetic radiation is only emitted when the finger is present (such as when pressure is detected on the pressure sensor).

In yet another alternative example, the synchronization process can be achieved by performing a cross-correlation between the one or more pressure sensors and the detected electromagnetic signals. When the signals are sufficiently broadband, the relative delays can be accurately measured. Various techniques can be employed to ensure that the signals are of a sufficient broadband. In at least one example, the device can provide video or audio feedback to the user via the communication unit to prompt the user to adjust the rate at which the user is pressing their finger to the pressure sensor (for example, the pressure or duration of with which the user contacts their finger with the device); thus assuring the generation of broadband signals. In another example, instead of relying on the user's application of pressure on the finger cradle, the device can be configured to generate a broadband signal using one or more of a buzzer, a voice coil, a piezoelectric actuator, or any other actuator capable of generating mechanic motion or adjusting a pressure.

In addition to informing the user of their hydration state, the device can also be configured to provide additional benefits to the user either directly or indirectly related to a user's hydration state. For example, the device can be configured to improve a user's sleep by informing whether his/her hydration level is at a predetermined level, such as sufficiently hydrated, before going to bed. In another example, the device can be configured to assist in a user's athletic performance by informing the user whether they are well hydrated before an athletic event, a training session, a workout, or any other athletic activity. In addition, the device can be used to improve a user's attention span by informing the user whether they are well hydrated. Furthermore, the device can assist a user in achieving his/her weight loss and/or mental awareness goals by informing the user of his/her hydration status. In addition, the device can assist a user in meeting his/her wellness, beauty, and/or skin care goals by informing them when it would be beneficial to drink more. Such benefits can, in at least one example, be achieved by the device sending a notification via the communication unit to instruct the user to perform a hydration measurement at predetermined points in time. Next, the device can use temporal trends to predict the change user dehydration rate over a defined period of time. For example, the device can use the predicted dehydration rate to determine when a user's hydration state will fall below a predetermined threshold which can be set based on a desired activity (i.e., certain hydration levels can be set for certain activities). Finally, the device can inform the user via the communication unit either when the user should consume fluids and how much fluid the user should drink.

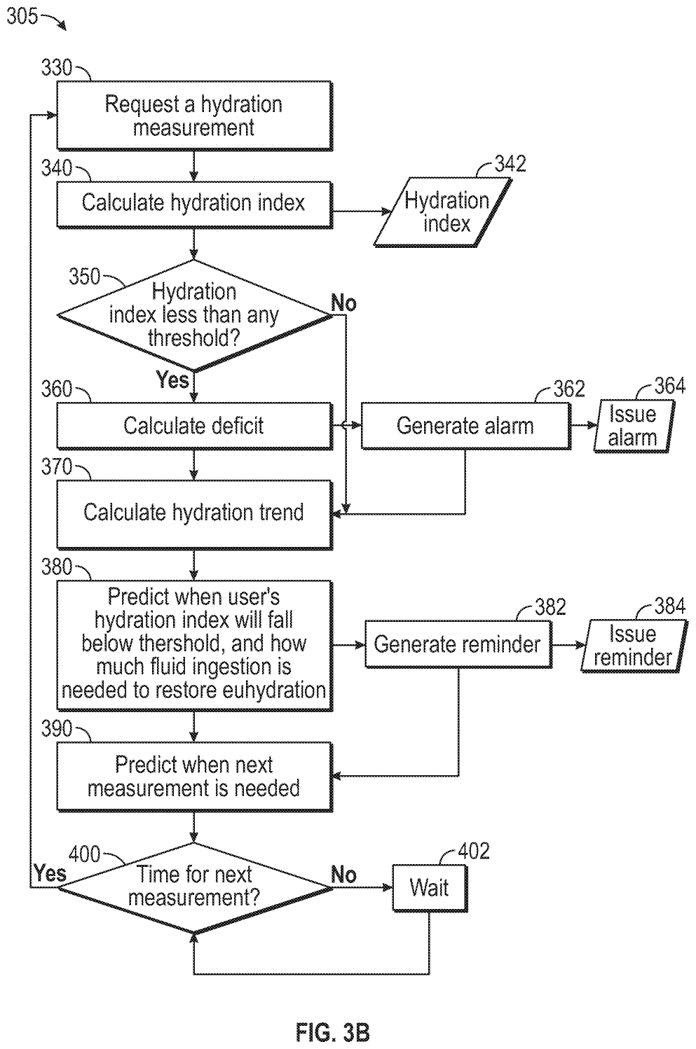

At least one example of the decision-making process performed by the processing unit to assist users in meeting his/her wellness goals is depicted in FIGS. 3A and 3B. Specifically, FIG. 3A is a flow chart illustrating a method 300 for requesting a user to perform a hydration measurement, and FIG. 3B is a flow-chart illustrating a detailed decision-making process 305 performed by the device to assist a user in attaining his/her goals.

With respect to FIG. 3A, the method 300 can start at block 310 by sending a request from the device to a user. The request can include a query for demographic information including, but not limited to, age, gender, weight, height, and combinations thereof. The request can also include a query for the user's wellness goals, such goals can include, but are not limited to, weight loss, mental alertness, skin care, and combinations thereof. Based on the user input of demographic data and goals, the method can progress to block 320 wherein the device can retrieve hydration threshold levels. For example, at block 322, the device can consult a library, the library can include a database of minimum hydration index levels which can be required in order to meet the user-defined goals. In at least one example, the library can be stored in an internal, non-volatile memory located within the device. In an alternative example, the library can be stored remotely and the device can be configured to access the library via wired or wireless connection. In at least one example, when the library is stored remotely, the device can be configured to access the library via an online server using the communication unit coupled with the device. In an additional example, when the library is stored remotely, the demographic data from any given user can be used to populate an online database of users, thus facilitating the use of machine learning methods in order to extract better hydration index thresholds based on a larger user population, which can then be used to benefit all users.

Once the device determines the relevant hydration data from the library, the method can proceed to block 330. At block 330, the device can request the user perform a hydration measurement. In at least one example, this measurement can be the first hydration measurement. In the alternative, the measurement can be a subsequent measurement. In at least one alternative example not illustrated in FIG. 3A, the requested measurement can be performed before the user enters his/her demographic information and wellness goals.

FIG. 3B illustrates an alternative method 305 for assisting a user attain his/her goals. Specifically, the method 305 can begin at block 330, wherein a user performs a hydration measurement using the device described above. At block 340, the device can collect data relating to the hydration measurement provided via a sensor as described above, and can use the data in order to calculate a new hydration index. At block 342, the information can be stored in a hydration index database. In at least one example, the information can be stored in a memory 126 integral to the device, as illustrated with respect to FIG. 1. The method 305 can then proceed to block 350, wherein the device can compare the hydration index resulting from the hydration measurement to hydration thresholds. The hydration thresholds can be goal-based hydration thresholds related to the activities, health, or wellness goals provided by the user. If the method 305 determines that the user hydration index is below any of the hydration thresholds, the method 305 can proceed to block 360 where a deficit is calculated. The deficit can indicate, for example, the difference between a goal-based hydration level (or desired hydration level) and the actual hydration level of a user. As such, the deficit can indicate a dehydration level of the user. Next, at block 362, the device can generate an alarm indicating the dehydration level of the user based on the calculated hydration deficit. At block 364, the device can issue an alarm via a communication device coupled with the device indicating the user can be required to take one or more actions. For example, the alarm can indicate one or more states including, but not limited to, whether the user should consume fluids, the recommended volume of fluid required to bring the user to a state within the threshold, the recommended volume of fluids required to restore the user to state of euhydration, and any other related information. In at least one example, a secondary threshold can be set such that an alarm is only generated if the dehydration level exceeds the secondary threshold.

Next, at block 370, the device can compare the current hydration indices against previous hydration measurements taken in order to estimate the user's dehydration rate. If the measurement taken in block 330 is a first measurement, then the user demographic data can be used to estimate the user's nominal dehydration rate. As measurements are repeated, the dehydration rate can be continuously updated in order to increase the accuracy of the calculations based on each individual user. For example, an adaptive filter can be used in conjunction with the method 305; the adaptive filter can include, but is not limited to, a Wiener filter, Least-mean squares filter, and/or a Kalman filter.

At block 380 the device can predict, using the dehydration rate determined above, the next point in time when the user hydration index could fall below the user-generated hydration threshold. Additionally, the method 305 can estimate the volume of fluid necessary to bring the user from his/her dehydrated state back to a euhydrated state. The device can then generate a reminder, such as, as a calendar event. At block 384, the reminder can be issued via the communication unit of the device informing the user how much fluid they should consume and when to consume it.

Next, the method 305 can proceed to block 390, where the device can estimate when the next hydration measurement is needed in order to increase the accuracy of the hydration index and hydration trend. In at least one example, the prediction can be performed by tracking the error covariance using a Kalman filter and performing additional hydration measurements for the user until the uncertainty level is reduced and the covariance falls below a desired threshold. As an illustrative example, the error covariance can be initially estimated as 0.1 and the desired threshold can be set as 0.05. The desired threshold can be determined, for example, based on a user input. In an alternative example, the desired threshold can be preset in the device. Once the method predicts when the next hydration measurement is needed, the method 305 can proceed to block 400. At block 400, the device can determine whether the time for the next hydration measurement has arrived. If not, the device can wait until the time for the next measurement arrives, as described in block 402. When the device determines it is time for the next measurement, the method 305 can proceed to block 330, the device indicate to the user that it is time to perform the next hydration measurement. After which, the method 305 can be repeated as necessary.

In the alternative, if the device determines at block 350 that the hydration index is not less than the user-generated hydration threshold, method 305 can proceed directly to block 370 and can compare the hydration index to the previous hydration measurements and calculate the user's hydration trend. The method 305 can then proceed to block 380 to predict when user's hydration index may next fall below the user-generated threshold and proceed as described above.

Other than wellness goals, the portable hydration monitoring device described herein can also be used in a clinical setting to assist health care providers. For example, the hydration monitoring device can be used to assist practitioners in the fluid management for their patients. The device can additionally be used to assist the practitioners in a variety of ways including, but not limited to, keeping a patient's homeostatic balance (such as by diagnosing conditions including, but not limited to, hypervolemia and hypovolemia), assisting in the management of a patient's proper blood volume, managing a patient's inputs and outputs (I/Os), as well as various other medical issues. In an additional example, the device described herein can be used in an Emergency Room setting in order to check for signs of acute and/or chronic dehydration, determine the volume of fluids to be administered (orally or intravenously) to bring the patient back to euhydration and homeostatic equilibrium, and the like. In yet another example, the device can be used by anesthesiologists in an operating room setting in order to help determine a patient's fluid needs during a procedure, or by a clinician in an intensive care unit to help ensure that patients are kept within a safe margin from their dehydration threshold. Moreover, skin perfusion can be affected by the blood pressure of the patient. As such, the device described herein can also be used to diagnose hypertension and/or hypotension. In yet another example, the device described herein can be used to promote renal protection by assuring a proper fluid level within the body of a user. The device described herein can also be used to reduce the risk of low blood pressure events by monitoring a user's hydration state and, the device can also assist in the prevention of damaging effects which can be a consequence of low blood pressure events, such as falls.

In addition to the above, the device can be used to assist in the care of the elderly. For example, elderly patients can progressively lose their ability to determine their own dehydration level by thirst drive alone. As such, the elderly can especially benefit from a device capable of providing them with reminders regarding when their hydration levels should be monitored, when to consume fluids, and how much fluid to consume. Furthermore, a portable hydration monitoring device can allow patients to be discharged from hospital care more quickly by enabling the home monitoring of patient hydration levels after certain procedures. Moreover, a portable hydration monitoring device can be helpful in preventing the re-hospitalization of patients who have been hospitalized or have undergone a medical procedure, considerably reducing health care costs and benefitting both patients and health care providers alike.

In another example, the device can be used to assist the military and/or first responders. A portable hydration monitoring device such as that described herein can be highly effective at detecting dehydration prior to the onset of heat injury and fatigue prior to musculoskeletal injury (MSI). For example, according to the Medical Surveillance Monthly Report (MSMR) there were 2,887 heat injuries in 2010 alone. Furthermore, a reported 14,018 heat injuries occurred between 2006-2010 with 25.7% of those cases being reported from two Army posts, Ft. Bragg and Ft. Benning. Another recent report indicated that 34% of medical evacuations in Operation Iraqi Freedom (OIF) and Operation Enduring Freedom (OEF) were the direct result of MSI—more than twice as many as those for combat injury.

As an additional example of military applicability, the device described herein can find application in the theater of war. For example, in a medical tent the device described herein can be used by medical officers to diagnose dehydration in patients suspected of MSI or of heat stroke at the end of a march. In addition, the device can be used to confirm whether soldiers have effectively consumed fluids at the required rate, which can replace the prevalent technique of forced fluid consumption.

Additionally, first responders are often required to perform extraneous physical activity in unsatisfactory conditions. For example, firemen combating a fire inside a building engulfed in flames typically lose large amounts of fluids in a relatively short period of time, in which case the device described herein can be used to warn the firemen when they need to consume a certain volume of fluids while on duty. First responders can also use the device to diagnose victims when they arrive on location. Moreover, the device can be used to monitor laborers performing heavy labor in warm locations. For example, construction workers working under direct Sun exposure in tropical locations, in which case the risk of heat stroke is increased even in the presence of small levels of dehydration.

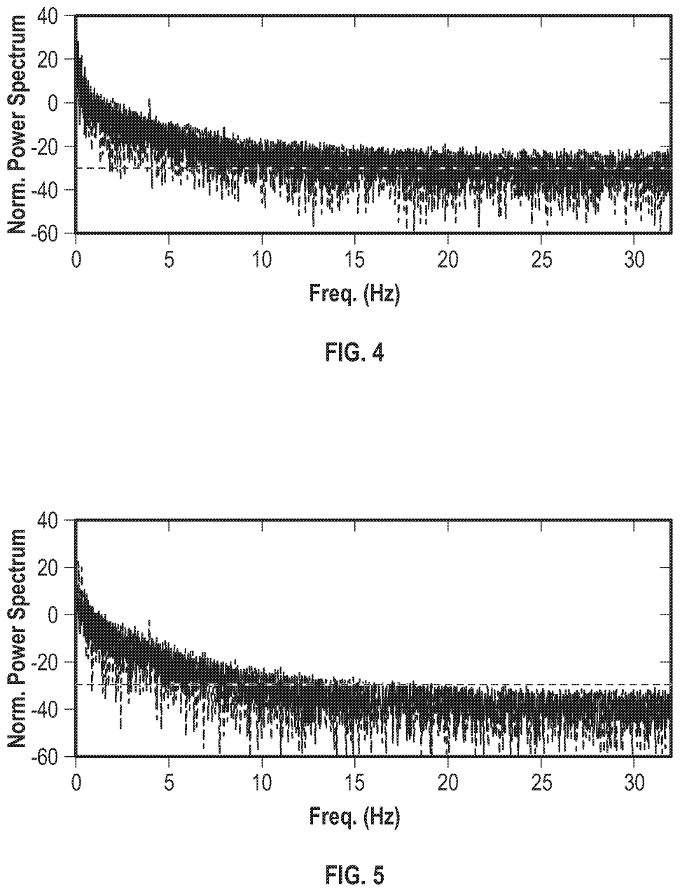

FIG. 4 illustrates a graph of the power spectral density (PSD) of electromagnetic radiation detected from a plurality of finger presses on a portable hydration monitoring device, such as that described above, in a euhydrated user. In the present example, the data was obtained when the user pressed his/her thumb to a hydration monitoring device multiple times; the device recorded electromagnetic signals resulting from the finger presses. The electromagnetic signals were then zero-padded to assure a constant minimum length. The padded signals were then squared, transformed using a Fourier analysis, and then converted into power signals which were normalized with respect to a standard deviations. Finally, the signals were converted into log space (decibels—dB), producing the plot shown in FIG. 4. In comparison, FIG. 5 illustrates a graph of the PSD from a plurality of finger presses from the same user after they have performed a workout that resulted in 1.97% of fluid loss, measured with respect to his euhydrated nude body weight (NBW).

As described above, the blood flow into and out of the capillaries through the finger can vary. This flow, however, may not be uniform and the temporal response can vary as a function of the hydration state of the user. Namely, when the user is more hydrated, blood flows more rapidly, resulting in stronger power components in the user's power spectral density (PSD), especially in the high frequency range of the spectrum, corresponding to the faster response times. Thus, the amplitude of the PSD at the higher range of the spectrum (for example, greater than or equal to 15 Hz) can be used to determine the hydration state of the user.

As shown in FIG. 4, in the euhydrated state the PSD at 15 Hz is about −20 dB (as indicated by the dashed horizontal line), while FIG. 5 shows that in the dehydrated state the PSD at 15 Hz is about −30 dB (as indicated by the dashed horizontal line). As shown, there is about a 10 dB drop in power corresponding to a 1.97% drop in the NBW hydration of the user.

In an alternative method, the change in spectrum can be measured more precisely by measuring the transfer function of the biological tissue being monitored. Namely, by capturing the spectrum of the finger press pressure along with the spectrum of the detected electromagnetic radiation waveform, the ratio of the two can be calculated, thus obtaining a normalized transfer function. Then, as described above, the changes in PSD power at a certain frequency range (for example, greater than or equal to 15 Hz) can be used to determine changes in the hydration state of a user. The alternative method presents an advantage in that it is less sensitive to variations in the user finger press activity. The device described herein can also issue an alarm, as described above, if the device detects that the user has pressed his/her finger too slowly for accurate detection, providing the user with appropriate feedback, such as an indication to increase the pace and/or speed of finger presses.

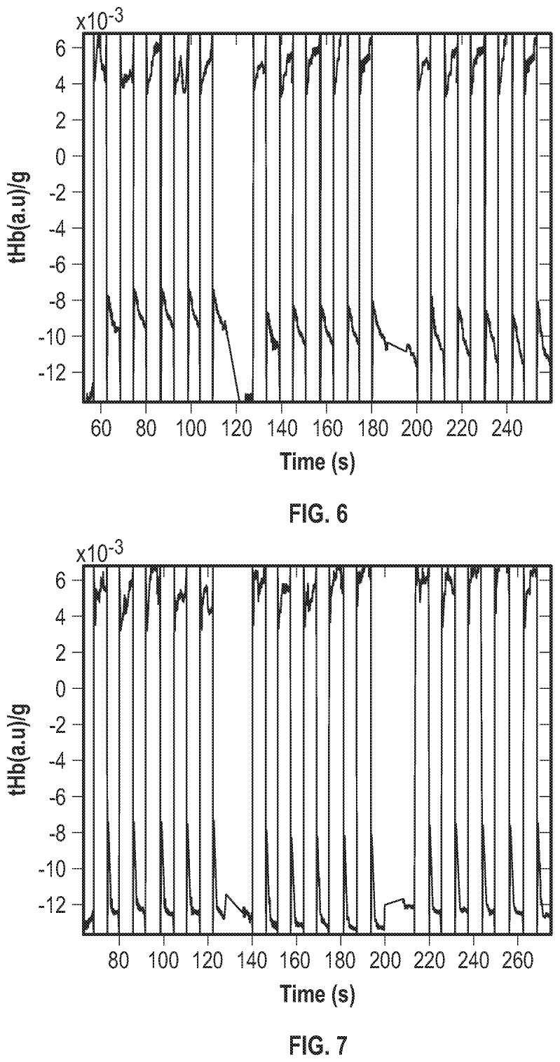

Yet another method to determine a user's hydration state from finger presses is illustrated in FIG. 6. In the present example, the skin turgor defined as the ratio between tHb and the load detected for the pressure sensor (given in grams) is used to determine the hydration state. The tHb is calculated using the sum of the estimated oxyhemoglobin and deoxyhemoglobin concentrations, which are in their turn given by the product of the detected analogue-to-digital converter (ADC) counts from a Red and near infrared light and a pseudo-inverse matrix obtained from the hemoglobin absorption spectra. Note that since skin turgor is calculated by a ratio, a good synchronization is required (such as within about 1 ms) between the electromagnetic radiation signal and the pressure signals.

For example, plot in FIG. 6 shows three sequences containing five finger presses each, of a euhydrated user. During each sequence, the finger pressure is varied from strong (for example, stronger than arterial) to light (for example, the finger is lightly touching the device). As illustrated, the point in time having a high finger pressure corresponds to the low plateaus in the data; specifically, the high pressure corresponds to low tHb and high load, hence turgor is the lowest. Correspondingly, the light finger pressure points correspond to the high plateaus; specifically, the low pressure corresponds to high tHb and low load, hence turgor is the highest).

FIG. 7 shows a corresponding plot after the same user has undergone a workout which resulted in the loss of bodily fluids corresponding to a dehydration of 1.97% with respect to his/her euhydrated NBW. As shown, the high turgor for the user points remained at the same level, around 5 tHb/g, while the low turgor points for the user dropped from an average level of about −10 tHb/g to about −13 tHb/g. As such, FIGS. 6-7 illustrate that the low turgor points during finger presses can be used to determine the hydration stage of a given user.

In addition, the hydration state of a user can be measured by using different definitions of turgor. In at least one example, skin turgor can be defined as the ratio of HbO₂ over the applied finger pressure. In an alternative example, skin turgor can be defined as the ratio of HHb over the applied finger pressure.

In additional implementations, the device can also use a bioimpedance signal as the source of the electromagnetic radiation signal instead of light. In such an example, the skin turgor can be given by the ratio of the bioimpedance over the applied pressure.

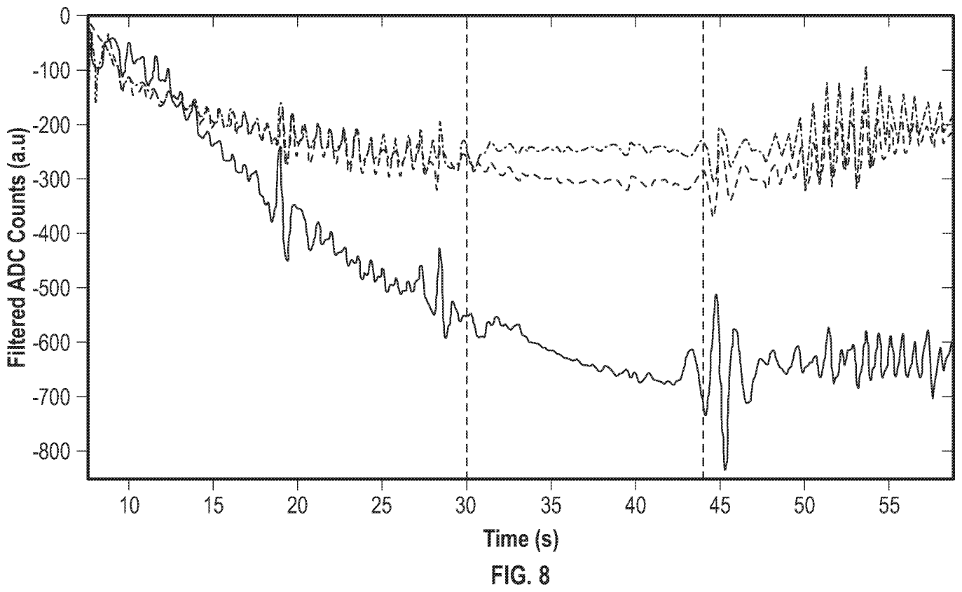

FIG. 8 illustrates yet another method to determine the hydration state of a user based on a plurality of finger presses. The data provided in the graph of FIG. 8 was collected using a prototype device and, in the present case the device was configured to evaluate the presence and absence of a variation in the photoplethysmograph (PPG) of the finger of a user. Namely, the volume of blood vessels, especially those located in extremities, vary as a function of the pressure wave produced by heart as it pumps arterial blood throughout the body's circulatory system. As blood volume increases, so does the absorption of light, resulting in temporal variations which are synchronous with the heart rate of the user. However, when the pressure applied to the tissue being monitored (in the present case, a finger) is increased beyond the arterial pressure, the tissue is no longer able to vary its volume as a function of heart beats. As a result, the amplitude variations in the PPG disappear, leading to a clear indication that the finger is pressed. Likewise, when the pressure is removed the variation returns, and the speed at which the return takes place is a direct measure of capillary refill time and, thus, of hydration.

The first dashed line indicated in FIG. 8, at around 30 seconds, shows the point in time when the finger of a user is pressed to the device. Note that the detected signals present clear amplitude variations before that point in time in all three wavelengths of light, including IR (950 nm peak, indicated herein by a dash-dotted line), red (650 nm peak, indicated herein by a dashed line), and green (540 nm peak, indicated herein by a solid line). After the finger is sufficiently pressed, however, the PPG amplitude is gone. This increase in finger pressure can be confirmed by the pressure sensor or by an IMU unit coupled with the device. The second dashed line, at around 44 seconds, shows the point in time when the finger pressure is released but the finger remains in contact (for example, light pressure) with the finger cradle, allowing for continued monitoring, including the detection of the return of the PPG amplitude.

In the example illustrated in FIG. 8, green light can also be used, emitted by a third LED with a spectrum centered at the wavelength of 540 nm. Green light is especially absorbed by hemoglobin, allowing the easy detection of heart rate peaks within a small volume of tissue.

The plot in FIG. 8 also illustrates that the device can be effective at acquiring PPG signals from a user's finger. As such, the device can also be used to monitor a patient non-invasively through other metrics that are commonly measured using PPG signals, including via heart rate, heart rate variability (HRV), and the activation of the sympathetic versus parasympathetic nervous system, including their uses in athletic training, detection of athletic overtraining and the management of training in athletes. Moreover, the PPG signal can also be used to measure the respiration rate and blood pressure of users.

In addition, the PPG signal can be used as an indicator of whether or not the user is placing his/her finger correctly on the finger cradle. For example, the device can provide the user with feedback, via the communication unit, on the adjusting the placement of the user's finger until the device detects a PPG signal with a sufficiently high amplitude. Conversely, the device can also detect, via the pressure sensor, the presence of a finger and provide the user with feedback on the correct finger placement via the communications unit.

The device can further include a thermometer capable of measuring the skin temperature of the finger of the user. Such thermometer can be useful in detecting when the skin temperature is low and a PPG signal is not present, thus indicating a potential for vasoconstriction. In the present example, the device can issue an alarm to the user, via the communication unit, informing the user to warm up his/her hand.

Additionally, the graph illustrated in FIG. 8 demonstrates that the device can effectively measure the user arterial pressure. For example, the device can use the pressure sensor to track arterial pressure, since the pressure reading is obtained at the time when the PPG amplitude disappears (or reappears) which corresponds to when the user is applying more (or less) pressure.

The stroke volume (SV) of a user can vary as a function of the user's hydration state. For example, when a user becomes less hydrated the user's SV can also be reduced. Since the cardiac output (CO) of the user can be determined by the product of the SV and the heart rate, the device can be configured to measure the user's SV and CO.

Furthermore, the device can comprise an electrocardiogram (EKG) unit configured to take measurements from the user. The device can then use the time delay between the EKG pulses and the PPG pulses to determine the pulse transit time (PTT) from the heart to the extremity (such as the finger), since the PTT is directly proportional to the arterial compliance and thus inversely proportional to the blood pressure. This can provide yet another method to monitor blood pressure via the device disclosed herein.

The above disclosure states that any of the five fingers can be used to perform the above described measurements. However, it should be noted that of all fingers, a user's thumb is most affected by volume changes in a photoplethysmogram. As such, the thumb can be especially useful in portable, non-invasive hydration monitoring. However, measurements taken using the index finger can also be beneficial, because users of portable devices are typically conditioned to using their index finger when interacting with touch screens and fingerprint readers.

The embodiments shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the embodiments described above may be modified within the scope of the appended claims.

1. A device comprising:
at least one pressure sensor;
a processing unit coupled to the at least one pressure sensor;
at least one emitter of electromagnetic radiation coupled to the processing unit;
at least one detector of electromagnetic radiation coupled to the processing unit;
at least one analog-to-digital converter coupled to the processing unit;
a memory coupled to the processing unit;
a synchronization line coupled to the processing unit;
a finger cradle coupled to the synchronization line; and
at least one alarm unit coupled to the processing unit.

2. The device of claim 1, wherein the device is integrated within at least one of a wearable device; a smartphone; a cellphone; a watch; a tablet; a laptop; a computer; and/or combinations thereof.

3. The device of claim 1, wherein the at least one alarm unit is operable to provide an alert selected from the group comprising a visual alarm, an audible alarm, a display indicating a user's hydration state, an audible message indicating a user's hydration state, and combinations thereof.

4. The device of claim 3, wherein the visual alarm indicates whether the finger presses are sufficiently broadband.

5. The device of claim 1, wherein the pressure sensor is selected from the group comprising a pressure plate, a capacitive force sensor, a piezoelectric sensor, and combinations thereof.

6. The device of claim 1, further comprising an inertial measurement unit (IMU) selected from the group comprising an accelerometer, a gyroscope, a motion sensor, and combinations thereof.

7. The device of claim 1, wherein the synchronization line encompasses at least one of: a trigger signal between the at least one pressure unit and the processing unit;
a trigger signal between the at least one emitter of electromagnetic radiation and the processing unit; and
a trigger signal between the at least one pressure unit, the at least one emitter of electromagnetic radiation, and the processing unit.

8. The device of claim 1, wherein the device is operable to perform one or more tasks selected from the group comprising optimizing a user's hydration, improving a user's sleep, improving a user's workout, improving a user's athletic performance, improving a user's attention span, assisting a user attain weight loss goals, assisting a user improve wellness, assisting a user attain beauty, assisting a user attain skincare related goals, and combinations thereof.

9. The device of claim 1, wherein the device is operable for one or more medical applications selected from the group comprising keeping a patient's homeostatic balance; managing a patient's inputs; managing a patient's outputs; promoting renal protection; assessing risk of low blood pressure events; assessing risk of falls due to low blood pressure events; managing a patient's blood volume; diagnosing hypervolemia; diagnosing hypovolemia; checking for acute dehydration; checking for chronic dehydration; diagnosing hypertension; diagnosing hypotension; and/or combinations thereof.

10. The device of claim 1, wherein the device is operable to prevent medical issues selected from the group comprising dehydration, heat stroke, and combinations thereof.

11. The device of claim 1, wherein the device is operable to measure values selected from the group comprising a photoplethysmograph (PPG), a heart rate, a heart rate variability, a respiratory rate, a blood pressure, an activation of a sympathetic nervous system, an activation of a parasympathetic nervous system, an overtraining in athletes, a management of athletic training, a stroke volume, a cardiac output, and combinations thereof.

12. The device of claim 1, further comprising one or more pressure waves generated by an element selected from the group comprising one or more buzzers, a piezoelectric actuator, a voice coil, a mechanic actuator, and combinations thereof.

13. The device of claim 1, further comprising an EKG operable to measure at least one of a pulse transit time (PTT), a blood pressure, and combinations thereof.

14. A method for monitoring hydration comprising:
pressing a finger of a user against a finger cradle of a device, creating a finger press;
sensing, via at least one sensor, a finger presence;
providing an alarm indicating a pressure to apply;
emitting, via at least one electromagnetic emitter, radiation through the finger;
detecting, via at least one electromagnetic detector, radiation through the finger;
verifying, via a synchronization line, that the electromagnetic radiation is applied synchronously with one or more finger presses;
converting, via at least one analog-to-digital converter, the radiation detected by the electromagnetic detector into digital information; and
converting, via a processing unit, finger pressure data and detected electromagnetic radiation data into a hydration level using an algorithm stored on the processing unit; and
conveying the hydration level to at least one alarm unit coupled with the device.

15. The method of claim 14, wherein the algorithm is operable to convert the electromagnetic radiation data into a chromophore concentration selected from the group comprising oxyhemoglobin concentration, deoxyhemoglobin concentration, total hemoglobin concentration, and combinations thereof.

16. The method of claim 14, wherein calculation of the hydration level is performed using a process selected from the group comprising:
a measurement of time delay between the one or more finger press and a change in detected electromagnetic radiation;
a measurement of a change in spectrum of the detected electromagnetic radiation;
a measurement of a change in the transfer function of the one or more finger presses, wherein the transfer function is given by a ratio of a spectrum of the pressure data and the detected electromagnetic radiation data;
a measurement of a turgor, wherein the turgor is given by a ratio of tHb and a pressure load;
measurement of a turgor, wherein the turgor is given by a ratio of HbO₂ and a pressure load;
a measurement of a turgor, wherein the turgor is given by a ratio of HHb and a pressure load;
a measurement of a turgor, wherein the turgor is given by a ratio of bioimpedance and a pressure load; and combinations thereof.

17. The method of claim 14, wherein the finger is a thumb.

18. The method of claim 14, wherein the finger is an index finger.

19. The method of claim 14, wherein the electromagnetic radiation includes at least one of an optical radiation in the wavelength range from about 600 nm to about 1000 nm and an electrical pulse in the range from about 100 Hz to about 250 MHz.

20. A device comprising:
at least one pressure sensor used to measure the pressure applied by at least one finger;
at least one emitter of electromagnetic radiation used to emit electromagnetic radiation into the at least one finger;
at least one detector of electromagnetic radiation used to detect electromagnetic radiation transmitted through the at least one finger;
at least one analog-to-digital converter used to digitize the electromagnetic radiation detected by the at least one detector of electromagnetic radiation;
a processing unit operable to process the data digitized by the analog-to-digital converter;
a memory, used by the processing unit to store data and execute algorithms;
a synchronization line;
a finger cradle used to place the at least one finger; and at least one alarm unit.
